(19) United States
(12) Patent Application Publication
(10) Pub. No.: US 2017/0176469 A1
(43) Pub. Date: Jun. 22, 2017

(54) Molecular Probes for Detecting Lipids

(71) Applicant: Case Western Reserve University, Cleveland, OH (US)

(72) Inventor: Yanming Wang, Beachwood, OH (US)

(21) Appl. No.: 14/380,822

(22) PCT Filed: Feb. 25, 2013
(86) PCT No.: PCT/US2013/027667

§ 371 Date: Aug. 25, 2014

Related U.S. Application Data
(60) Provisional application No. 61/602,988, filed on Feb. 24, 2012.

(51) Int. Cl. G01N 33/92; G01N 33/52

(52) U.S. Cl. CPC G01N 33/92; G01N 33/52; G01N 2800/044

(57) ABSTRACT

A method of detecting aberrant lipid accumulation in a subject includes administering to the subject a molecular probe that includes a fluorescent trans stilbene derivative or stilbenzene derivative and detecting the amount or distribution of the molecular probe in a tissue of interest of the subject.

### RELATED APPLICATION

This application claims priority from U.S. Provisional Application No. 61/602,988, filed Feb. 24, 2012, the subject matter of which is incorporated herein by reference in its entirety.

### TECHNICAL FIELD

This application relates to molecular probes and to their use in methods of detecting lipids in a subject, and particularly relates to molecular probes that can readily be used to detect aberrant lipid accumulation in a subject.

### BACKGROUND

Lipid storage diseases, or the lipidoses, are a group of inherited metabolic disorders in which harmful amounts of fatty materials (lipids) accumulate in various cells and tissues in the body. People with these disorders either do not produce enough of one of the enzymes needed to break down (metabolize) lipids or they produce enzymes that do not work properly. Over time, this excessive storage of fats can cause permanent cellular and tissue damage, particularly in the brain, peripheral nervous system, liver, spleen, and bone marrow.

Gaucher disease is the most common of the lipid storage diseases. It is caused by a deficiency of the enzyme glucocerebrosidase. Fatty material can collect in the spleen, liver, kidneys, lungs, brain, and bone marrow. Symptoms may include enlarged spleen and liver, liver malfunction, skeletal disorders and bone lesions that may cause pain and fractures, severe neurologic complications, swelling of lymph nodes and (occasionally) adjacent joints, distended abdomen, a brownish tint to the skin, anemia, low blood platelets, and yellow spots in the eyes. Persons affected most seriously may also be more susceptible to infection. The disease affects males and females equally.

Niemann-Pick disease is a group of autosomal recessive disorders caused by an accumulation of fat and cholesterol in cells of the liver, spleen, bone marrow, lungs, and, in some patients, brain. Neurological complications may include ataxia, eye paralysis, brain degeneration, learning problems, spasticity, feeding and swallowing difficulties, slurred speech, loss of muscle tone, hypersensitivity to touch, and some corneal clouding.

Farber's disease, also known as Farber's lipogranulomatosis, describes a group of rare autosomal recessive disorders that cause an accumulation of fatty material in the joints, tissues, and central nervous system. The disorder affects both males and females. Disease onset is typically in early infancy but may occur later in life. Children who have the classic form of Farber's disease develop neurological symptoms within the first few weeks of life. These symptoms may include moderately impaired mental ability and problems with swallowing. The liver, heart, and kidneys may also be affected. Other symptoms may include vomiting, arthritis, swollen lymph nodes, swollen joints, joint contractures (chronic shortening of muscles or tendons around joints), hoarseness, and xanthemas which thicken around joints as the disease progresses.

The gangliosidoses are comprised of two distinct groups of genetic diseases. Both are autosomal recessive and affect males and females equally. The GM1 gangliosidoses are caused by a deficiency of the enzyme beta-galactosidase, resulting in abnormal storage of acidic lipid materials particularly in the nerve cells in the central and peripheral nervous systems. The GM2 gangliosidoses also cause the body to store excess acidic fatty materials in tissues and cells, most notably in nerve cells. These disorders result from a deficiency of the enzyme beta-hexosaminidase.

The GM2 disorders include Tay-Sachs disease (also known as GM2 gangliosidosis-variant B) and Sandhoff disease (variant AB). Tay-Sachs and its variant forms are caused by a deficiency in the enzyme hexosaminidase A. Affected children appear to develop normally for the first few months of life. Symptoms begin by 6 months of age and include progressive loss of mental ability, dementia, decreased eye contact, increased startle reflex to noise, progressive loss of hearing leading to deafness, difficulty in swallowing, blindness, cherry-red spots in the retinas, and some paralysis. Seizures may begin in the child's second year. A rarer form of the disorder, called late-onset Tay-Sachs disease, occurs in patients in their twenties and early thirties and is characterized by unsteadiness of gait and progressive neurological deterioration. Sandhoff disease (variant AB) usually occurs at the age of 6 months and is not limited to any ethnic group. Neurological signs may include progressive deterioration of the central nervous system, motor weakness, early blindness, marked startle response to sound, spasticity, myoclonus (shock-like contractions of a muscle), seizures, macrocephaly (an abnormally enlarged head), and cherry-red spots in the eye.

Krabbé disease (also known as globoid cell leukodystrophy and galactosylceramide lipidosis) is an autosomal recessive disorder caused by deficiency of the enzyme galactocerebrosidase. The buildup of undigested fats affects the growth of the nerve's protective myelin sheath and causes severe deterioration of mental and motor skills. Other symptoms include muscle weakness, hypertonia (reduced ability of a muscle to stretch), myoclonic seizures (sudden, shock-like contractions of the limbs), spasticity, irritability, unexplained fever, deafness, optic atrophy and blindness, paralysis, and difficulty when swallowing. Prolonged weight loss may also occur.

Metachromatic leukodystrophy, or MLD, is a group of disorders marked by storage buildup in the white matter of the central nervous system and in the peripheral nerves and to some extent in the kidneys. Similar to Krabbé disease, MLD affects the myelin that covers and protects the nerves. This autosomal recessive disorder is caused by a deficiency of the enzyme arylsulfatase A. Both males and females are affected by this disorder.

Wolman's disease, also known as acid lipase deficiency, is a severe lipid storage disorder that is usually fatal by age 1. This autosomal recessive disorder is marked by accumulation of cholesteryl esters (normally a transport form of cholesterol) and triglycerides (a chemical form in which fats exist in the body) that can build up significantly and cause damage in the cells and tissues. Both males and females are affected by this disorder. Infants are normal and active at birth but quickly develop progressive mental deterioration, enlarged liver and grossly enlarged spleen, distended abdomen, gastrointestinal problems including steatorrhea (excessive amounts of fats in the stools), jaundice, anemia, vomiting, and calcium deposits in the adrenal glands, causing them to harden.

Fabry disease is a glycosphingolipid (GSL) lysosomal storage disorder resulting from an X-linked inherited deficiency of lysosomal α-galactosidase A (α-GAL), an enzyme responsible for the hydrolysis of terminal α-galactosyl residues from glycosphingolipids. A deficiency in the enzyme activity results in a progressive deposition of neutral glycosphingolipids, predominantly globotriaosylceramide (ceramide trihexoside, CTH, GL-3), in cell of Fabry patients. Symptoms can be severe and debilitating, including kidney failure and increased risk of heart attack and stroke. Certain of the mutations cause changes in the amino acid sequence of α-GAL that may result in the production of α-GAL with reduced stability that does not fold into its correct three-dimensional shape. Although α-GAL produced in patient cells often retains the potential for some level of biological activity, the cell's quality control mechanisms recognize and retain misfolded α-GAL in the endoplasmic reticulum, or ER, until it is ultimately moved to another part of the cell for degradation and elimination. Consequently, little or no α-GAL moves to the lysosome, where it normally hydrolyzes GL-3. This leads to accumulation of GL-3 in cells, which is believed to be the cause of the symptoms of Fabry disease. In addition, accumulation of the misfolded α-GAL enzyme in the ER may lead to stress on cells and inflammatory-like responses, which may contribute to cellular dysfunction and disease.

### SUMMARY

Embodiments described herein relate to a method of detecting aberrant lipid accumulation in a tissue of interest of a subject. The method includes administering to the tissue of interest of the subject a molecular probe, which includes a fluorescent trans-stilbene derivative or stilbenzene derivative. The amount and/or distribution of the molecular probe in the tissue of interest of the subject is then detected. The amount and/or distribution of the detected molecular probe in the tissue of interest is indicative of the amount and/or distribution of aberrant lipid accumulation in the tissue of interest.

In some embodiments, the molecular probe can include a compound selected from the group consisting of

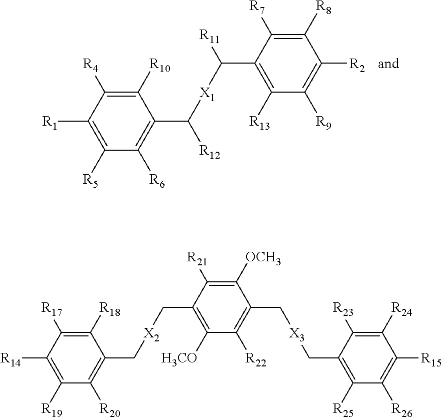

wherein R₁, R₂, R₁₄ and R₁₅ are each independently a hydrophilic or lipophilic group; wherein each X₁, X₂, and X₃ is a double or triple bond; each R₄-R₁₃ and R₁₇-R₂₆ is independently selected from the group consisting of H, F, Cl, Br, I, a lower alkyl group, an alkylene group, an alkenyl group, an alkynyl group, an alkoxy group, an aryl group, an aryloxy group, an alkaryl group, an aralkyl group, O, (CH₂)ₙOR′ (wherein n=1, 2, or 3), CF₃, CH₂—CH₂X, O—CH₂—CH₂X, CH₂—CH₂—CH₂X, O—CH₂—CH₂X (wherein X═H, F, Cl, Br, or I), CN, C═O, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group, wherein R′ is H or a substituted or unsubstituted lower alkyl group); wherein R₁₀ and R₁₁ and/or R₁₂ and R₁₃ may be linked to form a cyclic ring, wherein the cyclic ring is aromatic, alicyclic, heteroaromatic, or heteroalicyclic; or a pharmaceutically acceptable salt thereof.

In other embodiments, the molecular probe can further include a radiolabel. The radiolabel can include at least one of a ³H, ¹²⁵I, ¹¹C, or ¹⁸F. The molecular probe can also include a chelating group or a near infrared imaging group.

In some embodiments, X₁ can be a double bond. R₁, R₂, R₁₄, and R₁₅ can be amines or alkyl derivatives thereof. R₄-R₁₃ and R₁₇-R₂₆ can be H.

In some embodiments, the aberrant lipids that are accumulated can include sphingolipids. The subject can have or can be at risk of a lipid storage disorder. The lipid storage disorder can include Fabry disease.

In still other embodiments, an agent, such as a therapeutic agent for treating aberrant lipid accumulation, can be administered to the subject prior to administering the molecular probe to the subject. The detected amount and/or distribution of the molecular probe in the tissue of interest can be compared to a control amount and/or distribution. A decrease in the detected amount and/or distribution of the molecular probe in the tissue of interest is indicative of the agent reducing the aberrant lipid accumulation in the tissue of interest. The agent administered to the subject can be screened for efficacy in treating aberrant lipid accumulation associated with a lipid storage disease. The lipid storage disease can be Fabry disease.

Embodiments described herein also relate to a method of determining the efficacy of an agent in reducing sphingolipid accumulation in a tissue of interest of a subject. The method includes administering the agent to the subject. A molecular probe, which includes a fluorescent trans-stilbene derivative or stilbenzene derivative, is also administered to the subject. The amount or distribution of the molecular probe in the tissue of interest is then detected. The detected amount and/or distribution of the molecular probe in the tissue of interest can be compared to a control amount and/or distribution. A decrease in the detected amount and/or distribution of the molecular probe in the tissue of interest is indicative of the agent reducing the sphingolipids in the tissue of interest.

In some embodiments, the compound can be selected from the group consisting of

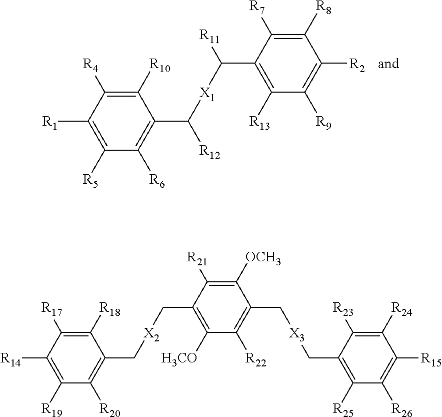

wherein R₁, R₂, R₁₄ and R₁₅ are each independently a hydrophilic or lipophilic group; wherein each X₁, X₂, and X₃ is a double or triple bond; each R₄-R₁₃ and R₁₇-R₂₆ is independently selected from the group consisting of H, F, Cl, Br, I, a lower alkyl group, an alkylene group, an alkenyl group, an alkynyl group, an alkoxy group, an aryl group, an aryloxy group, an alkaryl group, an aralkyl group, O, (CH₂)ₙOR′ (wherein n=1, 2, or 3), CF₃, CH₂—CH₂X, O—CH₂—CH₂X, CH₂—CH₂—CH₂X, O—CH₂—CH₂X (wherein X═H, F, Cl, Br, or I), CN, C═O, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group, wherein R′ is H or a substituted or unsubstituted lower alkyl group); wherein R₁₀ and R₁₁ and/or R₁₂ and R₁₃ may be linked to form a cyclic ring, wherein the cyclic ring is aromatic, alicyclic, heteroaromatic, or heteroalicyclic; or a pharmaceutically acceptable salt thereof.

In some embodiments, the subject has or is at risk of a lipid storage disorder. In other embodiments, the lipid storage disorder can be Fabry disease.

### BRIEF DESCRIPTION OF THE DRAWINGS

The foregoing and other features and advantages will become apparent to those skilled in the art to which this application relates upon reading the following description with reference to the accompanying drawings.

FIG. 1 depicts six representative structures of proposed sphingolipid (e.g., GL-3) binding agents evaluated for in vivo PET imaging.

FIG. 2 illustrates CIC fluorescent staining of rental tubular epithelial cells in wild-type and GLA KO mouse kidneys. (A). CIC staining of wild-type mouse kidney showing no specific accumulation in the rental tubular cells. (B). CIC staining of GL-3 deposition present in GLA knockout mouse kidney showing specific accumulation in the rental tubular that is consistent with immunohistochemistry (C).

FIG. 3 illustrates a series of microPET images showing left (top line) and right (bottom line) kidneys of wild-type rat after intravenous injection of [¹¹C]CIC.

FIG. 4 illustrates autographical images of wild-type and GLA knockout mouse kidney tissue sections after incubation with [¹¹C]AIC showing significantly higher uptake of [¹¹C]AIC in the GLA KO kidneys with GL-3 deposition.

FIG. 5 illustrates a series of coronal PET images of a wild-type rat showing [¹¹C]AIC uptake in the kidneys. Both left and right kidneys can be clearly visualized at early time points with fast clearance due to lack of GL-3 deposition.

### DETAILED DESCRIPTION

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “Fabry disease” refers to an X-linked inborn error of glycosphingolipid catabolism due to deficient lysosomal α-galactosidase A activity. This defect causes accumulation of globotriaosylceramide (ceramide trihexoside) and related sphingolipids in vascular endothelial lysosomes of the heart, kidneys, skin, brain, and other tissues.

The term “atypical Fabry disease” refers to patients with primarily cardiac manifestations of the α-GAL deficiency, namely progressive globotriaosylceramide (GL-3) accumulation in myocardial cells that leads to significant enlargement of the heart, particularly the left ventricle.

A “carrier” is a female who has one X chromosome with a defective α-GAL gene and one X chromosome with the normal gene and in whom X chromosome inactivation of the normal allele is present in one or more cell types. A carrier is often afflicted with Fabry disease.

A “patient” or “subject” refers to a subject who has been diagnosed with a particular disease. The patient may be human or animal. A “Fabry disease patient” refers to an individual who has been diagnosed with Fabry disease and has a mutated α-GAL as defined further below. Characteristic markers of Fabry disease can occur in male hemizygotes and female carriers with the same prevalence, although females typically are less severely affected.

Human α-galactosidase A (α-GAL) refers to an enzyme encoded by the human Gla gene. The human α-GAL enzyme consists of 429 amino acids and is in GenBank Accession No. U78027.

As used herein in one embodiment, the term “mutant α-GAL” includes an α-GAL, which has a mutation in the gene encoding α-GAL that results in the inability of the enzyme to achieve a stable conformation under the conditions normally present in the ER. The failure to achieve a stable conformation results in a substantial amount of the enzyme being degraded, rather than being transported to the lysosome. Such a mutation is sometimes called a “conformational mutant.”

Non-limiting, exemplary α-GAL mutations associated with Fabry disease, which result in unstable α-GAL include L32P; N34S; T41I; M51K; E59K; E66Q; I91T; A97V; R100K; R112C; R112H; F113L; T141L; A143T; G144V; S148N; A156V; L166V; D170V; C172Y; G183D; P205T; Y207C; Y207S; N215S; A228P; S235C; D244N; P259R; N263S; N264A; G272S; S276G; Q279E; Q279K; Q279H; M284T; W287C; I289F; M296I; M296V; L300P; R301Q; V316E; N320Y; G325D; G328A; R342Q; E358A; E358K; R363C; R363H; G370S; and P409A.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” derivative or analog, refers to a derivative or analog having the same type of pharmacological activity as the parent compound and approximately equivalent in degree.

As used herein, the term “pharmaceutically acceptable salts” or complexes refers to salts or complexes that retain the desired biological activity of the parent compound and exhibit minimal, if any, undesired toxicological effects. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acids, naphthalenedisulfonic acids, and polygalacturonic acid; (b) base addition salts formed with cations, such as sodium, potassium, zinc, calcium, bismuth, barium, magnesium, aluminum, copper, cobalt, nickel, cadmium, sodium, potassium, and the like, or with an organic cation formed from N,N-dibenzylethylene-diamine, ammonium, or ethylenediamine; or (c) combinations of (a) and (b); e.g., a zinc tannate salt or the like.

This application relates to molecular probes that can bind to lipids, such as sphingolipids (e.g., GL-3 or GB-3), that are associated with aberrant lipid accumulation in (or of) a cell, tissue, and/or organ of subject. The molecular probes can be used to detect aberrant lipid accumulation in a cell, tissue, and/or organ of interest of a subject. The tissue and/or organ can include, for example, the heart, kidney, or brain. The molecular probes can be administered to and/or provided with the cell, tissue, and/or organ of interest of a subject and upon administration bind to, localize with, and/or complex with the lipids in the cell, tissue, and/or organ. The molecular probes can then be detected using, for example, an imaging technique to determine or quantify the amount and/or distribution of the molecular probes and the associated aberrant lipid accumulation in the cell, tissue, and/or organ of interest of the subject.

In some embodiments, the lipids can be sphingolipids and/or glycosphingolipids and the aberrant lipid accumulation can be associated with a lipid storage disease or disorder, such as Gaucher disease, Niemann-Pick disease, Farber's disease, gangliosidoses, GM2 disorders, Krabbé disease, Metachromatic leukodystrophy, Wolman's disease, and Fabry disease. For example, the lipid can be a sphingolipid, such as globotriaosylceramide, that is formed as a result of a deficiency of the enzyme alpha galactosidase A, which is associate with Fabry disease, and the molecular probe upon administration to a tissue (kidneys, heart, skin, vasculature, brain etc.) of interest of a subject can be used to detect the amount and/or distribution of sphingolipid in the tissue of interest.

The molecular probes can be administered in vitro, ex vivo, in vivo to a cell, tissue, and/or organ of interest of the subject and be readily visualized using conventional visualization techniques to indicate lipid accumulation in the cell, tissue, or organ of the subject including the heart, kidneys, skin, brain, and/or vasculature of the subject. In some aspects, the molecular probes can be used in a method of detecting aberrant lipid accumulation in vivo in a subject. In other aspects, the molecular probes can be used in a method of detecting aberrant lipid accumulation associated with a lipid storage disease (e.g., Fabry disease). In still other aspects, the molecular probes can be used in a method of screening agents for inhibiting aberrant lipid accumulation associated with a lipid storage disease. In yet other aspects, the molecular probes can be used for measuring the efficacy of an agent or therapy in inhibiting aberrant lipid accumulation in a subject.

In some embodiments, the molecular probe can include a fluorescent trans-stilbene derivative or a pharmacophore thereof (e.g., coumarin pharmacophore) that is less than about 700 daltons and has a relatively high binding affinity to sphingolipids associated with lipid storage disease, such as GL-3.

In some embodiments, the fluorescent trans-stilbene derivative can have the formula:

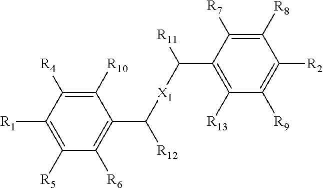

wherein R₁ and R₂ are each independently a hydrophilic or lipophilic group; wherein X₁ is a double or triple bond; each R₄-R₁₃ is independently selected from the group consisting of H, a halogen group (e.g., H, F, Cl, Br, I), a lower alkyl group, an alkylene group, an alkenyl group, an alkynyl group, an alkoxy group, an aryl group, an aryloxy group, an alkaryl group, an aralkyl group, O, (CH₂)ₙOR′ (wherein n=1, 2, or 3), CF₃, CH₂—CH₂X, O—CH₂—CH₂X, CH₂—CH₂—CH₂X, O—CH₂—CH₂X (wherein X═F, Cl, Br, or I), CN, C═O, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group, wherein R′ is H or a substituted or unsubstituted lower alkyl group); wherein R₁₀ and R₁₁ and/or R₁₂ and R₁₃ may be linked to form a cyclic ring, wherein the cyclic ring is aromatic, alicyclic, heteroaromatic, or heteroalicyclic; or a pharmaceutically acceptable salt thereof.

The phrase “have the formula” or “having the structure” is not intended to be limiting and is used in the same way that the term “comprising” is commonly used.

The term “alkyl” refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups, such as cyclopentyl, cyclohexyl, and the like. Generally, although again not necessarily, alkyl groups herein contain 1 to about 18 carbon atoms, preferably 1 to about 12 carbon atoms. The term “lower alkyl” intends an alkyl group of 1 to 6 carbon atoms. Substituents identified as “C₁-C₆ alkyl” or “lower alkyl” can contain 1 to 3 carbon atoms, and more particularly such substituents can contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “alkenyl” refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups can contain 2 to about 18 carbon atoms, and more particularly 2 to 12 carbon atoms. The term “lower alkenyl” refers to an alkenyl group of 2 to 6 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups can contain 2 to about 18 carbon atoms, and more particularly can contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” refers to an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Preferred substituents identified as “C₁-C₆ alkoxy” or “lower alkoxy” herein contain 1 to 3 carbon atoms, and particularly preferred such substituents contain 1 or 2 carbon atoms (i.e., methoxy and ethoxy).

The term “aryl” refers to an aromatic substituent containing a single aromatic ring or multiple aromatic rings that are fused together, directly linked, or indirectly linked (such that the different aromatic rings are bound to a common group such as a methylene or ethylene moiety). Aryl groups can contain 5 to 20 carbon atoms, and particularly preferred aryl groups can contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 20 carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Exemplary aralkyl groups contain 6 to 24 carbon atoms, and particularly preferred aralkyl groups contain 6 to 16 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like.

The term “cyclic” refers to alicyclic or aromatic substituents that may or may not be substituted and/or heteroatom containing, and that may be monocyclic, bicyclic, or polycyclic.

The terms “halo” and “halogen” are used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.”

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

In some embodiments, R₁ and/or R₂ can be selected from the group consisting of H, NO₂, NH₂, NHCH₃, N(CH₃)₂, OH, OCH₃, COOCH₃, SH, SCH₃, alkyl derivatives thereof, substituted alkyl derivatives thereof, and each R₄-R₁₃ is H.

In one example, the molecular probe can include a fluorescent stilbene derivative having the formula:

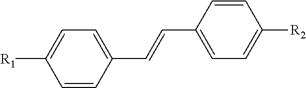

wherein R₁ and R₂ are each independently selected from the group consisting of H, NO₂, NH₂, NHCH₃, N(CH₃)₂, OH, OCH₃, COOCH₃, SH, SCH₃, alkyl derivatives thereof, substituted alkyl derivatives thereof, or a pharmaceutically acceptable salt thereof.

In a further aspect, the molecular probe can be selected from the following general structures:

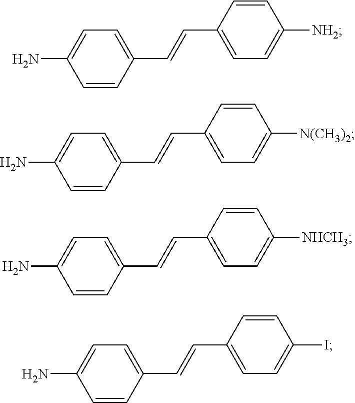

or pharmaceutically acceptable salts thereof.

In another embodiment, the molecular probe can include a fluorescent coumarin derivative that is a pharmacophore of trans-stilbene. In an aspect of the invention, R₁ and R₂ are each independently selected from the group consisting of H, NO₂, NH₂, NHCH₃, N(CH₃)₂, OH, OCH₃, COOCH₃, SH, SCH₃, alkyl derivatives thereof, and substituted alkyl derivatives thereof, X₁ is a double bond, and R₁₀ and R₁₁ are linked to form a heterocyclic ring.

In one example, the fluorescent coumarin derivative can have the formula:

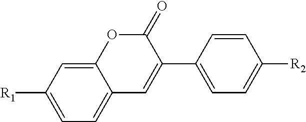

wherein R₁ and R₂ are each independently selected from the group consisting of H, NO₂, NH₂, NHCH₃, N(CH₃)₂, OH, OCH₃, COOCH₃, SH, SCH₃, alkyl derivatives thereof, and substituted alkyl derivatives thereof; and pharmaceutically acceptable salts thereof.

In another example, the fluorescent coumarin derivative can have the formula:

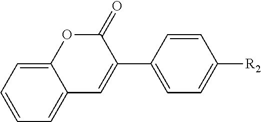

wherein R₁ and R₂ are each independently selected from the group consisting of H, NO₂, NH₂, NHCH₃, N(CH₃)₂, OH, OCH₃, COOCH₃, SH, SCH₃, alkyl derivatives thereof, substituted alkyl derivatives thereof, and pharmaceutically salts thereof.

In a further example, the fluorescent coumarin derivative can have the formula:

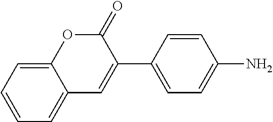 [SMILES: Nc1ccc(-c2cc3ccccc3oc2=O)cc1]

or a pharmaceutically acceptable salt thereof.

In a further example, the fluorescent coumarin derivative can have the formula:

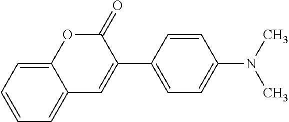 [SMILES: CN(C)c1ccc(-c2cc3ccccc3oc2=O)cc1]

or a pharmaceutically acceptable salt thereof.

In a further example, the fluorescent coumarin derivative can have the formula:

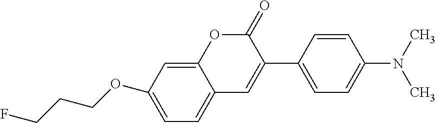 [SMILES: CN(C)c1ccc(-c2cc3ccc(OCCCF)cc3oc2=O)cc1]

or a pharmaceutically acceptable salt thereof.

In another embodiment, the molecular probe can be a fluorescent stilbenzene derivative that is less than about 700 daltons and has an excitation spectra at a wavelength of about 300 nm to about 500 nm (emission at 506 nm) and emission spectra upon exciting at a wavelength of about 430 nm to about 650 nm (excitation at 426 nm).

In some embodiments, the fluorescent stilbenzene derivative can have the formula:

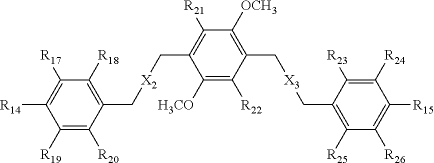

wherein R₁₄ and R₁₅ are each independently a hydrophilic or lipophilic group; wherein X₂ and X₃ are each independently a double or triple bond; and each R₁₇-R₂₆ is independently selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂X (wherein X═H, F, Cl, Br, or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group, wherein R′ is H or a substituted or unsubstituted lower alkyl group) or a pharmaceutically acceptable salt thereof. In some embodiments, R₁₄ and/or R₁₅ can be selected from the group consisting of H, NO₂, NH₂, NHCH₃, N(CH₃)₂, OH, OCH₃, COOCH₃, SH, SCH₃, alkyl derivatives thereof, and substituted alkyl derivatives thereof, and each R17-R₂₆ is H.

In one example, the fluorescent stilbenzene derivative can have the formula:

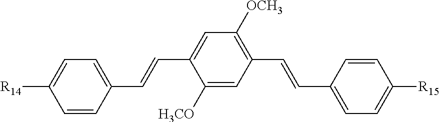

wherein R₁ and R₂ are each independently selected from the group consisting of H, NO₂, NH₂, NHCH₃, N(CH₃)₂, OH, OCH₃, COOCH₃, SH, SCH₃, alkyl derivatives thereof, and substituted alkyl derivatives thereof or a pharmaceutically acceptable salt thereof.

In another example, the fluorescent stilbenzene derivative can have the formula:

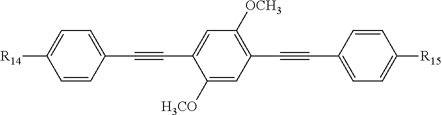

wherein R₁ and R₂ are each independently selected from the group consisting of H, NO₂, NH₂, NHCH₃, N(CH₃)₂, OH, OCH₃, COOCH₃, SH, SCH₃, alkyl derivatives thereof, and substituted alkyl derivatives thereof, or a pharmaceutically acceptable salt thereof.

In a further example, the fluorescent stilbenzene derivative can be (E,E)-1,4-bis(4′-aminostyryl)-2-dimethoxy-benzene (BDB), which has the general structure:

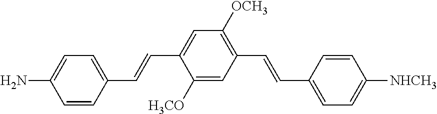 [SMILES: CNc1ccc(/C=C/c2cc(OC)c(/C=C/c3ccc(N)cc3)cc2OC)cc1]

or a pharmaceutically acceptable salt thereof.

In a still further example, the fluorescent stilbenzene derivative can be (E,E)-1,4-bis(4′-aminostyryl)-2-dimethoxy-benzene (BDB), which has the general structure:

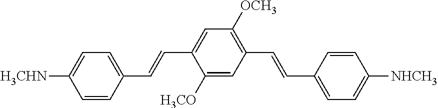

or a pharmaceutically acceptable salt thereof.

The foregoing formulae represent the general structures of molecular probes found to for labeling sphingolipids (e.g., GL-3) associated with lipid storage diseases (e.g., Fabry disease) in vivo as well as in vitro as described in the Example below. They are characterized by their ability to be administered to a mammal or subject parenterally and selectively localize to aberrant lipid accumulation in tissues or organs. The molecular probes are unique in that they exhibit negligible toxicities as demonstrated in both preclinical and clinical settings, making them candidates for clinical imaging modalities and translational studies. For example, once radiolabelled with positron-emitting radionuclide, they can be used for positron emission tomography to detect and quantify aberrant lipid accumulation in vivo.

Typically, the molecular probe can be formulated into pharmaceutical composition or solution prior to use. In one example, a molecular probe solution includes a 10 mM molecular probe solution. A molecular probe solution can also contain saline, DMSO, and HCL. One skilled in the art can utilize the molecular probe with pharmaceutical carriers and/or excipients in varying concentrations and formulations depending on the desired use.

In some embodiments, the molecular probe can be radiolabeled to aid in the detection of the molecular probe once it binds to myelin. A ‘radiolabel’ as used herein is any compound that has been joined with a radioactive substance. Examples of radiolabels include positron emitting ³H, ¹²⁵I, ¹¹C, and 18F radiolabels.

In some embodiments, the molecular probe can be coupled to a chelating group (with or without a chelated metal group) to improve the MRI contrast properties of the molecular probe. In one example, as disclosed in U.S. Pat. No. 7,351,401 which is herein incorporated by reference in its entirety, the chelating group can be of the form W-L or V-W-L, wherein V is selected from the group consisting of —COO—, —CO—, —CH₂O— and —CH₂NH—; W is —(CH₂)n where n=0, 1, 2, 3, 4, or 5; and L is:

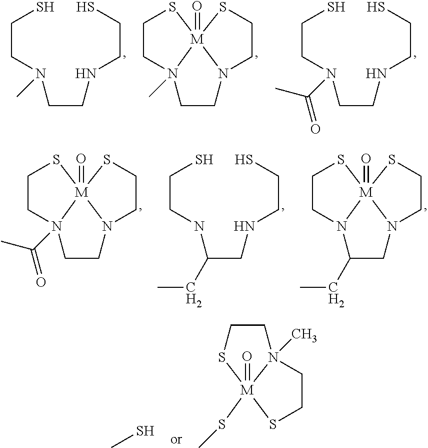

wherein M is selected from the group consisting of Tc and Re; or

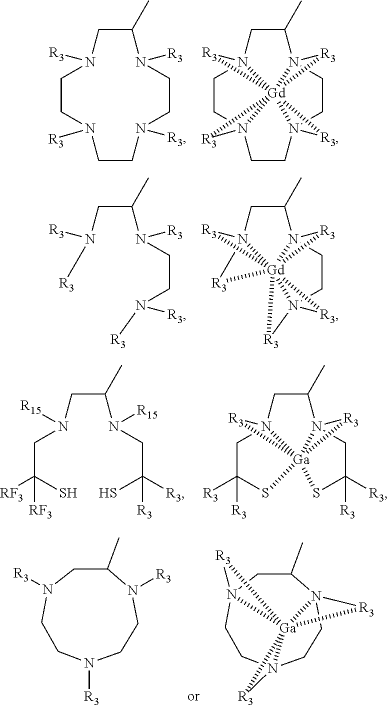

wherein each R₃ is independently is selected from one of:

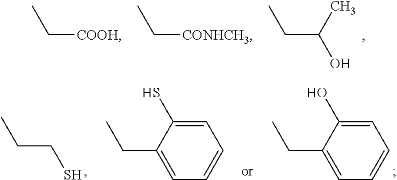

or a lipid binding, chelating compound (with or without a chelated metal group) or a water soluble, non-toxic salt thereof of the form:

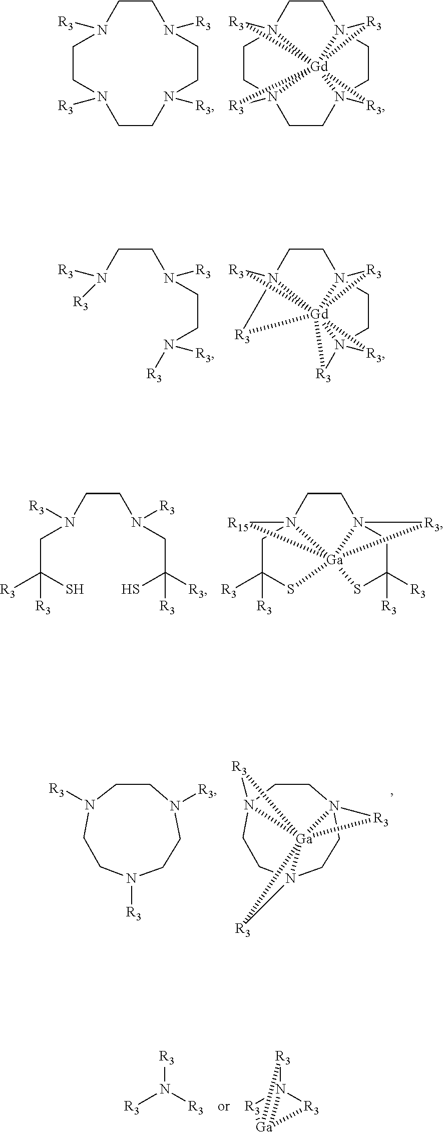

wherein each R₃ independently is selected from one of:

### H,

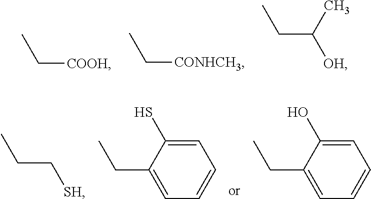

The chelating group can be coupled to at least one terminal benzene groups or the R₁, R₂, R₁₄, and/or R₁₅ group of the above noted compounds. In one example, the chelating group can be coupled to terminal amino R₁, R₂, R₁₄, and/or R₁₅ group through a carbon chain link. The carbon chain link can comprise, for example about 2 to about 10 methylene groups and have a formula of, for example, (CH₂)ₙ, wherein n=2 to 10.

In one example, the molecular probe with the chelating group can have the formula:

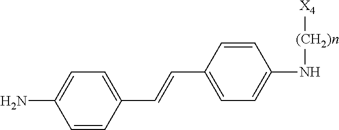

wherein X₄ is a chelating group and n is 2 to 10; or a pharmaceutically acceptable salt thereof.

In another example, the molecular probe with the chelating group can have the formula:

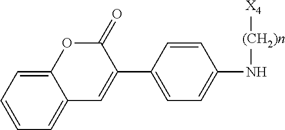

wherein X₄ is a chelating group and n is 2 to 10; or a pharmaceutically acceptable salt thereof.

In another example, the molecular probe with the chelating group can have the formula:

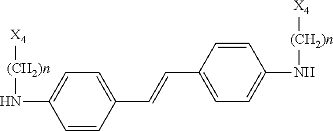

wherein X₃ is a chelating group and n is 2 to 10; or a pharmaceutically acceptable salt thereof.

In one example, the molecular probe with the chelating group can have the formula:

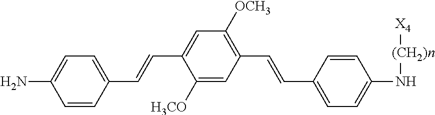

wherein X₄ is a chelating group and n is 2 to 10; or a pharmaceutically acceptable salt thereof.

In another example, the molecular probe can have the formula:

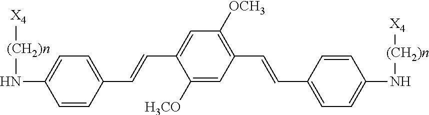

wherein X₄ is a chelating group and n is 2 to 10; or a salt thereof.

In a further example, the molecular probe with the chelating group can have the formula:

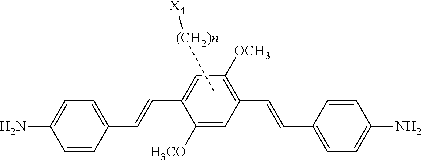

wherein X₄ is a chelating group and n is 2 to 10; or a pharmaceutically acceptable salt thereof.

In another embodiment, the molecular probe can be coupled to a near infrared group to improve the near infrared imaging of the molecular probe. Examples of near infrared imaging groups that can be coupled to the molecular probe include:

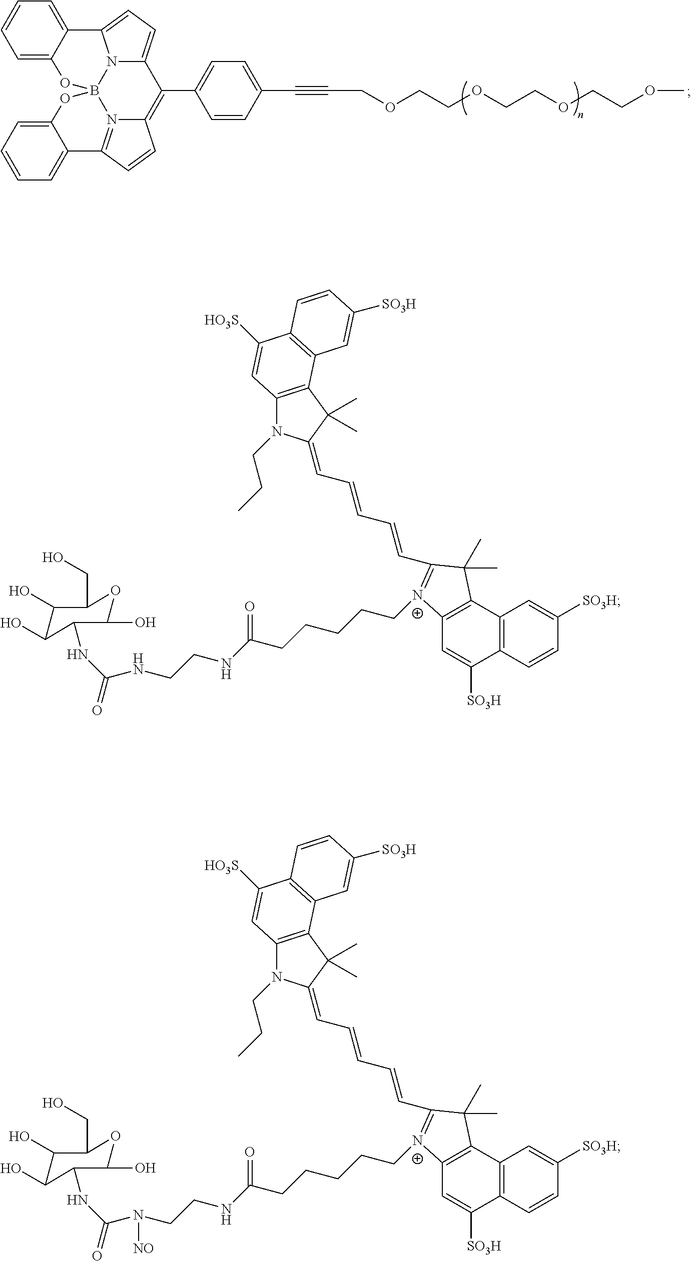

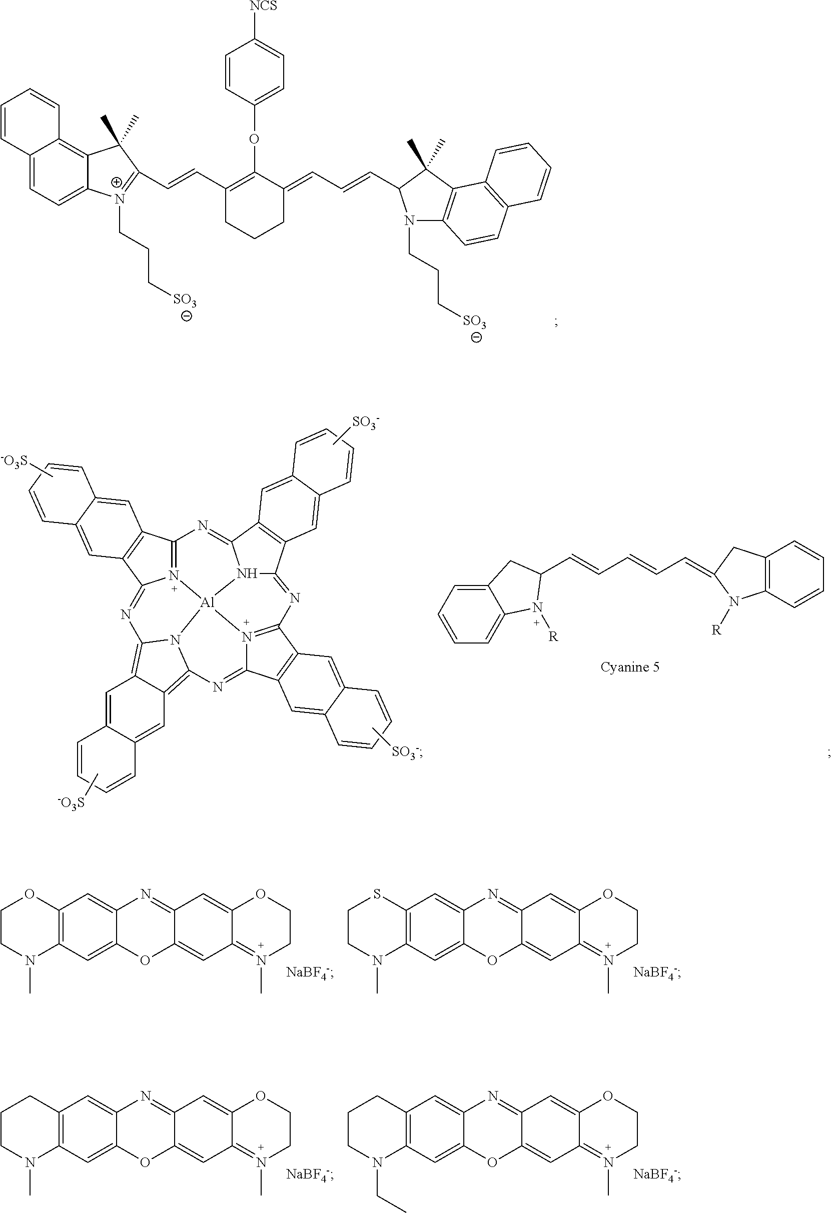

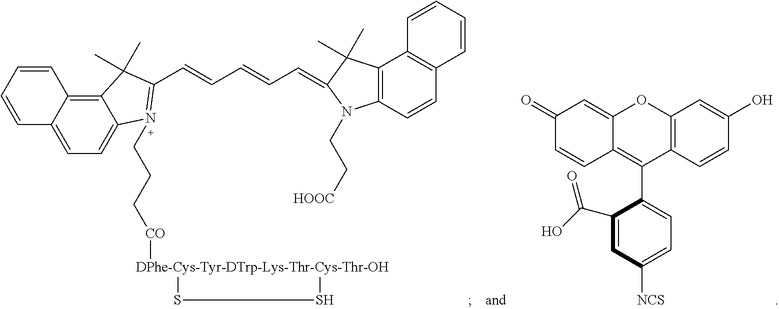

These near infrared imaging groups are disclosed in, for example, Tetrahedron Letters 49 (2008) 3395-3399; Angew. Chem. Int. Ed. 2007, 46, 8998-9001; Anal. Chem. 2000, 72, 5907; Nature Biotechnology vol 23, 577-583; Eur Radiol (2003) 13: 195-208; and Cancer 67: 1991 2529-2537, which are herein incorporated by reference in their entirety.

The near infrared imaging group can be coupled to at least one terminal benzene groups, or the R₁, R₂, R₁₄, or R₁₅ groups. In some embodiments, the near infrared imaging group can be coupled to at least one terminal benzene group.

In one example, the molecular probe with the near infrared imaging group can have the formula:

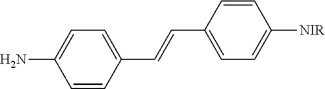

wherein NIR is a near infrared imaging group; or a pharmaceutically acceptable salt thereof.

In another example, the molecular probe with the near infrared imaging group can have the formula:

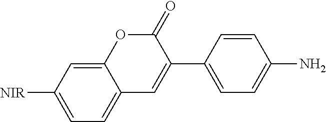

wherein NIR is a near infrared imaging group; or a pharmaceutically acceptable salt thereof.

By way of example, the molecular probe can include a compound having the

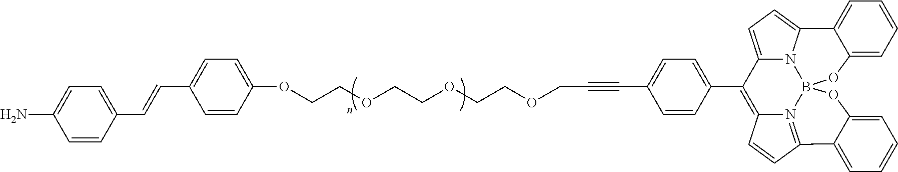

formula:
wherein n is 3 to 10; or a salt thereof.

In another example, the molecular probe with the near infrared imaging group can have the formula:

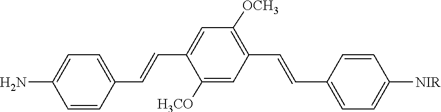

wherein NIR is a near infrared imaging group; or a pharmaceutically acceptable salt thereof.

By way of example, the molecular probe can include a compound having the formula:

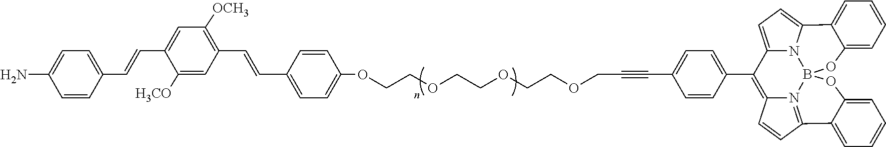

wherein n is 3 to 10; or a salt thereof.

In another example, the molecular probe can have the formula:

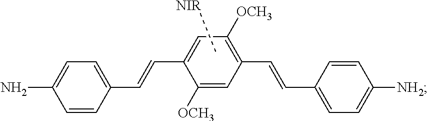

wherein NIR is a near infrared imaging group; or a pharmaceutically acceptable salt thereof.

In certain embodiments, the molecular probes can be administered to an animal and utilized for labeling and detecting lipidated regions of the animal's kidneys, skin, heart, brain and/or vasculature. In some embodiments, the molecular probes described herein can be used for the in vivo detection and localization of aberrant lipid accumulation of an animal's heart, kidneys, vasculature, brain, and skin. The molecular probe can be administered to the animal as per the examples contained herein, but typically through intravenous injection. The administration to the animal or subject can include providing or delivering molecular probes in an amount(s) and for a period of time(s) effective to label the lipid being detected. The molecular probes can be administered to the animal enterally or parenterally in, for example, a pharmaceutical composition that includes a solid or liquid. Enteral route includes oral, rectal, topical, buccal, and vaginal administration. Parenteral route includes intravenous, intramuscular, intraperitoneal, intrasternal, and subcutaneous injection or infusion.

The molecular probes described herein can be used for anatomical or pathological studies. Researchers studying aberrant lipid accumulation can employ these molecular probes in a method to examine the morphology and distribution of lipid accumulation in tissue or organs of interest of an animal or subject. “Distribution” as used herein is the spatial property of being scattered about over an area or volume. In this case the “distribution of lipid accumulation” is the spatial property of lipids being scattered about over an area or volume included in the animal's tissue or organs. Researchers interested in toxicology and pathology can also use this method in several ways. One way is to infer lipid accumulation by the presence of the molecular probe labeling compared to normal control tissue (e.g., normal kidneys). In yet another embodiment, one skilled in the art can assess and quantify changes in aberrant lipid accumulation in vivo.

In other aspects, aberrant lipid accumulation in an animal's tissue or organs, such as kidneys, heart, brain, or vasculature can be visualized and quantified using an in vivo imaging modality to visualize the molecular probes administered to the subject. The molecular probe may be visualized any time post administration depending on the application as typical molecular probes embodied herein have a low clearance rate due to specific binding or localizing in the regions including aberrant lipid accumulation.

An in vivo imaging modality as used herein is an imaging modality capable of visualizing molecular probes described herein in vivo (within a living organism). An example of an in vivo imaging modality is positron emission tomography (PET). PET is a functional imaging technique that can detect chemical and metabolic change at the molecular level. To function as a PET imaging molecular probe, embodiments of the molecular probe described herein must meet a set of biological requirements known to the skilled artisan, some of which may include lipophilicity, binding affinity, binding specificity, uptake, retention, and metabolism. Another example of an in vivo imaging modality is MicroPET. MicroPET is a high resolution positron emission tomography scanner designed for imaging small laboratory animals. Other examples of imaging modalities that can be used in accordance with the present invention include magnetic resonance imaging (MRI), near infrared (NIR) imaging, fluorescent microscopy, and multiphoton microscopy.

The molecular probes described herein can also be used to detect or diagnose aberrant lipid accumulation associated with a lipid storage disease in an animal through the use of in vivo lipid labeling. Thus, in certain embodiments, solutions containing the molecular probes described herein can be used in the detection of lipid storage diseases in an animal.

Methods of detecting aberrant lipid accumulation include the steps of labeling lipids, such as sphingolipids, ex vivo or in vivo in the animal's tissue with a molecular probe described herein, visualizing a distribution of the molecular probe in the animal's tissue as described above and in the examples, and then correlating the amount and/or distribution of the molecular probe with aberrant lipid accumulation in the animal. In one example, the methods described herein can be used to compare lipid accumulation in regions of the kidney in the normal tissues of control populations to those of a suspect animal. If the suspect animal has aberrant lipid accumulation, an increase in quantity and/or distribution of lipids may be present in the tissue thus indicating the presence of an aberrant lipid accumulation.

Another embodiment relates to a method of monitoring or measuring the efficacy of an agent or therapy in inhibiting aberrant lipid accumulation in a subject. The methods described herein include the steps of labeling lipids in vivo in the animal's tissue (e.g., kidneys) with a molecular probe described herein, then detecting, imaging, and/or visualizing the amount and/or distribution of the molecular probe in the animal's tissue (e.g., with an in vivo imaging modality as described herein), and then correlating the distribution of the molecular probe as visualized in the animal's tissue with the efficacy of the therapy or agent. It is contemplated that the labeling step can occur before, during, and after the course of a therapeutic regimen in order to determine the efficacy of the therapeutic regimen. One way to assess the efficacy of an agent or therapy is to compare the amount and/or distribution of the molecular probe before administration of the agent or therapy with the distribution of the molecular probe after therapy has commenced or concluded. A decrease in the detected amount and/or distribution of the molecular probe in the tissue of interest is indicative of the agent reducing the sphingolipids in the tissue of interest. An increase in the detected amount and/or distribution of the molecular probe in the tissue of interest is indicative of the agent having little or no effect in reducing the levels the sphingolipids in the tissue of interest.

Another embodiment relates to a method for screening agents for inhibiting aberrant lipid accumulation associated with a lipid storage disease. The method includes the initial step of administering an agent to an experimental animal that has or is at risk of aberrant lipid accumulation, such as a transgenic mouse model of Fabry disease. Lipid accumulation in the animal's tissue is labeled in vivo or in vitro with a molecular probe as described herein. An amount and/or distribution of the molecular probe in the animal's tissue can then visualized using a conventional visualization modality. Finally, the distribution of the molecular probe after the agents' response in the animal's tissue is correlated to the agent. One way to assess the agents' response in the animal's tissue is to compare the distribution of the molecular probe in an animal's tissue, which has been treated with a suspect agent with the distribution of the molecular probe in the tissue of a control population. The control population can be a population or a tissue sample not exposed to the agent under study but otherwise as close in all characteristics to the exposed group as possible. The molecular probes described herein can be used to determine if an agent of interest has the potential to modulate lipid accumulation (e.g., sphingolipid accumulation) of an experimental animal's tissue (e.g., kidneys) associated with a lipid storage disease.

### Example

We evaluated a series of small molecule probes as positron emission tomography (PET) agents for quantitative analysis of GL-3 deposition in the kidney. We found that some of the imaging agents can bind to GL-3 in vitro in the kidney of a transgenic mouse model of Fabry disease. In this Example, we determined the structural features of molecular probes that can be used for binding to GL-3 with high affinity and specificity. We then optimized the in vivo pharmacokinetic properties necessary for longitudinal monitoring GL-3 deposition in animal models of Fabry disease. The optimized molecular probes can then be radiolabeled and used as radiotracers for PET imaging and quantification of GL-3 in vivo. This allows us to develop an imaging tool for both preclinical drug screening in animal models and clinical evaluations of therapeutic treatments in Fabry disease patients. In the long term, it will enable us to apply the same imaging approach to study other lipid storage diseases.

### RESULTS

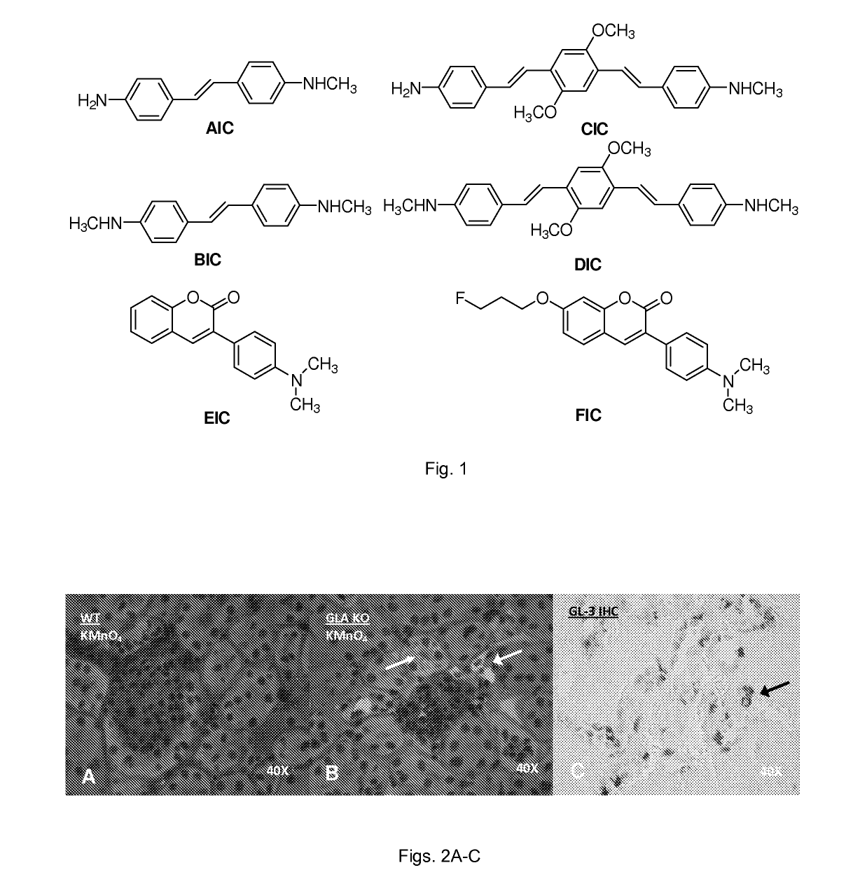

To date, we have identified some prototypical structures that can be used for GL-3 imaging. Some of the GL-3 imaging agents we have designed are shown in FIG. 1

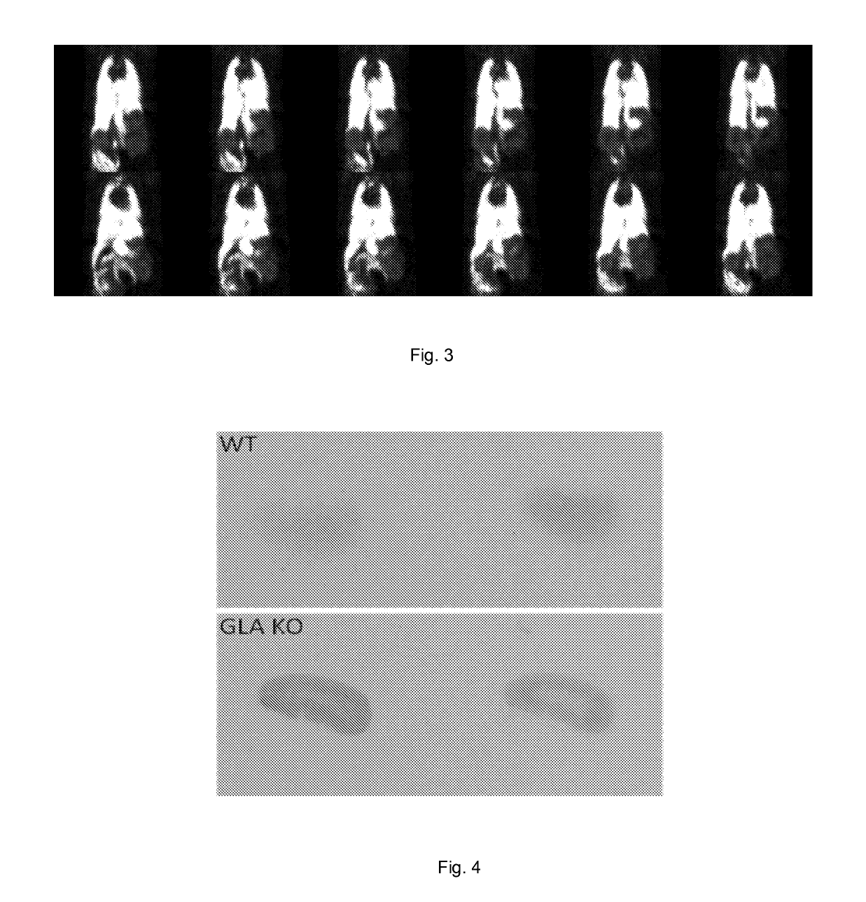

Given that some compounds are strongly fluorescent; we started evaluating its binding property by fluorescent tissue staining of kidney tissue sections of a transgenic mouse model of GL-3 deposition. We screened some compounds and preliminary results showed that a bis-stilbene derivative, termed CIC, exhibited high specificity for GL-3 as shown in FIG. 2. Tissue sections were first treated with KMₙO₄ to eliminate autofluorescence. Subsequent tissue staining using 2-10 mM of CIC showed that CIC was readily detected in renal tubular epithelial cells in GLA knockout mouse kidneys where GL-3 deposition has been reported. The staining pattern was found to be consistent with that of GL-3 immunohistochemistry.

Following fluorescent tissue staining, we radiolabeled CIC with C-11 and conducted whole body microPET studies in wild-type rats. This study was designed to show if [¹¹C]CIC can be used for PET imaging of the kidney. A series of coronal PET images are shown in FIG. 3, where both the right and left kidneys can be visualized. Since there was no significant GL-3 accumulation present in the wild-type kidney, only weak PET signals could be detected. Nonetheless, the kidneys can be readily visualized. This sets the stage for further structural optimization and in vivo PET imaging studies in a transgenic mouse model.

[¹¹C]CIC-PET images showed a relative high background in the abdomen region. To reduce the background, we further evaluated a monostilbene derivative termed AIC. AIC is almost half of the size of CIC. Thus, it may clear faster than CIC from other organs. Because AIC is not strongly fluorescent, its binding properties could not be readily assessed by fluorescent tissue staining. To circumvent this problem, we radiolabeled AIC with carbon-11 and conducted autoradiography using both wild-type and GLA knockout mouse kidney tissue sections. As shown in FIG. 4, [¹¹C]AIC accumulation in GLA knockout mouse kidney with GL-3 deposition is significantly higher than that in wild-type tissue sections.

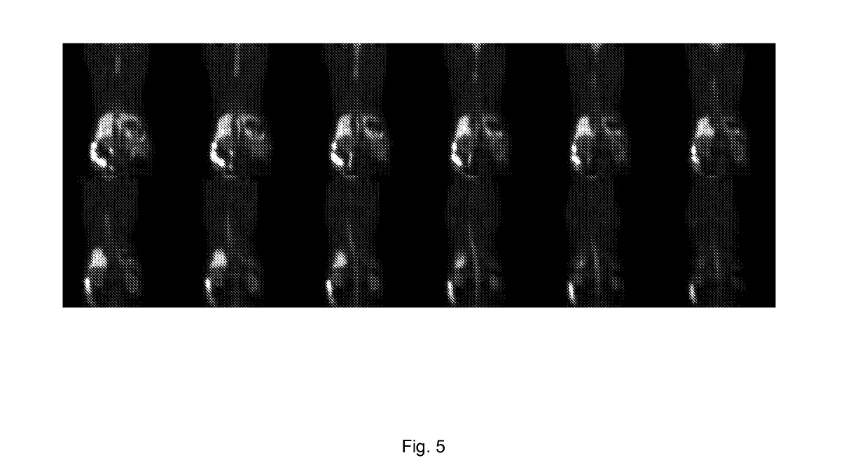

Encouraged by this result, we conducted in vivo [¹¹C]AIC-PET imaging in wild-type rats to determine its biodistribution relative to kidney uptake. As shown in FIG. 5, background of [¹¹C]AIC-PET images was significantly reduced compared to [¹¹C]CIC-PET images. In addition, [¹¹C]AIC was quickly cleared at later time points, which is expected due to the lack of GL-3 deposition.

The present invention is not to be limited in scope by the specific embodiments described herein. Indeed, various modifications of the invention in addition to those described herein will become apparent to those skilled in the art from the foregoing description and accompanying Figures. Such modifications are intended to fall within the scope of the appended claims. Various references are cited herein, the disclosure of which are incorporated by reference in their entireties.

1: A method of detecting aberrant lipid accumulation in a tissue of interest of a subject, the method comprising:
administering to the tissue of interest of the subject a molecular probe comprising a compound selected from the group consisting of: 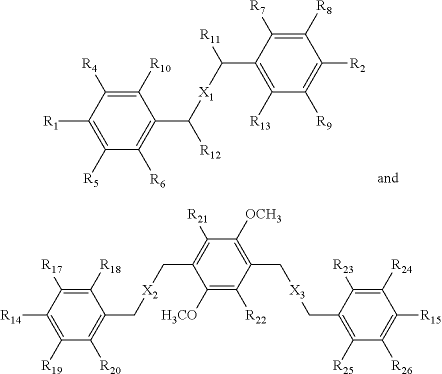
wherein R₁, R₂, R₁₄ and R₁₅ are each independently a hydrophilic or lipophilic group; wherein each X₁, X₂, and X₃ is a double or triple bond; each R₄-R₁₃ and R₁₇-R₂₆ is independently selected from the group consisting of H, F, Cl, Br, I, a lower alkyl group, an alkylene group, an alkenyl group, an alkynyl group, an alkoxy group, an aryl group, an aryloxy group, an alkaryl group, an aralkyl group, O, (CH₂)ₙOR′ (wherein n=1, 2, or 3), CF₃, CH₂—CH₂X, O—CH₂—CH₂X, CH₂—CH₂—CH₂X, O—CH₂—CH₂X (wherein X═H, F, Cl, Br, or I), CN, C═O, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group, wherein R′ is H or a substituted or unsubstituted lower alkyl group); wherein R₁₀ and R₁₁ and/or R₁₂ and R₁₃ may be linked to form a cyclic ring, wherein the cyclic ring is aromatic, alicyclic, heteroaromatic, or heteroalicyclic; or a pharmaceutically acceptable salt thereof; and
detecting the amount or distribution of the molecular probe in the tissue of interest of the subject, wherein the amount or distribution of the detected molecular probe in the tissue of interest is indicative of the amount or distribution of aberrant lipid accumulation in the tissue of interest.

2: The method of claim 1, the molecular probe of claim 1 further comprising a radiolabel.

3: The method of claim 2, the radiolabel including at least one of a ³H, ¹²⁵I, ¹¹C, or ¹⁸F.

4: The method of claim 1, the molecular probe further comprising a chelating group or a near infrared imaging group.

5: The method of claim 1, wherein X₁ is a double bond.

6: The method of claim 1, wherein R₁, R₂, R₁₄, and R₁₅ are amines or alkyl derivatives thereof.

7: The method of claim 1, wherein the aberrant lipids that are accumulated comprise sphingolipids.

8: The method of claim 1, wherein the subject has or is at risk of a lipid storage disorder.

9: The method of claim 1, wherein the lipid storage disorder comprises Fabry disease.

10: The method of claim 1, wherein the molecular probe is administered to the subject by parenteral administration.

11: The method of claim 1, further comprising administering an agent to the subject prior to administering the molecular probe to the subject and comparing the detected amount or distribution of the molecular probe in the tissue of interest to a control amount or distribution, wherein a decrease in the detected amount or distribution of the molecular probe in the tissue of interest is indicative of the agent reducing the aberrant lipid accumulation in the tissue of interest.

12: The method of claim 11, the agent administered to the subject being screened for efficacy in treating aberrant lipid accumulation associated with a lipid storage disease.

13: The method of claim 12, the lipid storage disease being Fabry disease.

14: A method of detecting sphingolipids in a tissue of interest of a subject, the method comprising:
administering to the subject a molecular probe comprising a compound selected from the group consisting of 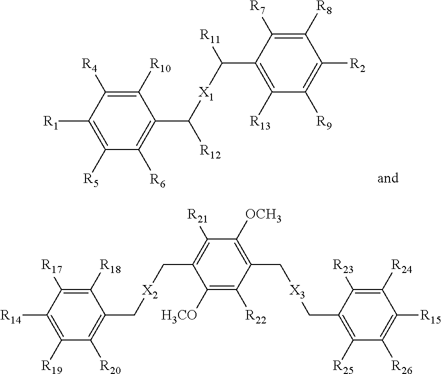
wherein R₁, R₂, R₁₄ and R₁₅ are each independently a hydrophilic or lipophilic group; wherein each X₁, X₂, and X₃ is a double or triple bond; each R₄-R₁₃ and R₁₇-R₂₆ is independently selected from the group consisting of H, F, Cl, Br, I, a lower alkyl group, an alkylene group, an alkenyl group, an alkynyl group, an alkoxy group, an aryl group, an aryloxy group, an alkaryl group, an aralkyl group, O, (CH₂)ₙOR′ (wherein n=1, 2, or 3), CF₃, CH₂—CH₂X, O—CH₂—CH₂X, CH₂—CH₂—CH₂X, O—CH₂—CH₂X (wherein X═H, F, Cl, Br, or I), CN, C═O, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group, wherein R′ is H or a substituted or unsubstituted lower alkyl group); wherein R₁₀ and R₁₁ and/or R₁₂ and R₁₃ may be linked to form a cyclic ring, wherein the cyclic ring is aromatic, alicyclic, heteroaromatic, or heteroalicyclic; or a pharmaceutically acceptable salt thereof; and
detecting the amount or distribution of the molecular probe in the tissue of interest, wherein the amount or distribution of the detected molecular probe in the tissue of interest is indicative of the amount or distribution of sphingolipids in the tissue of interest.

15: The method of claim 14, the molecular probe further comprising a radiolabel.

16: The method of claim 15, the radiolabel including at least one of a ³H, ¹²⁵I, ¹¹C, or ¹⁸F.

17: The method of claim 14, the molecular probe further comprising a chelating group or a near infrared imaging group.

18: The method of claim 14, wherein X₁ is a double bond.

19: The method of claim 14, wherein R₁, R₂, R₁₄, and R₁₅ are amines or alkyl derivatives thereof.

20: The method of claim 14, wherein the subject has or is at risk of a lipid storage disorder.

21: The method of claim 20, wherein the lipid storage disorder is Fabry disease.

22: The method of claim 14, wherein the molecular probe is administered to the subject by parenteral administration.

23: The method of claim 14, further comprising administering an agent to the subject prior to administering the molecular probe to the subject and comparing the detected amount or distribution of the molecular probe in the tissue of interest to a control amount or distribution, wherein a decrease in the detected amount or distribution of the molecular probe in the tissue of interest is indicative of the agent reducing the sphingolipids in the tissue of interest.

24: The method of claim 23, the agent administered to the subject being screened for efficacy in treating aberrant lipid accumulation associated with a lipid storage disease.

25: The method of claim 24, wherein the lipid storage disease is Fabry disease.

26-33. (canceled)
